(19) United States
(12) Patent Application Publication
(10) Pub. No.: US 2023/0225809 A1
(43) Pub. Date: Jul. 20, 2023

(54) ROBOTIC CUTTING GUIDE SYSTEM FOR COMPUTER-ASSISTED SURGERY

(71) Applicant: OMNIlife science, Inc., Raynham, MA (US)

(72) Inventors: Christopher Plaskos, Plymouth, MA (US); Sami Shalhoub, Braintree, MA (US); Frederic Leger, Rennes (FR); Christian Joly, Pleasanton, CA (US)

(73) Assignee: OMNIlife science, Inc., Raynham, MA (US)

(21) Appl. No.: 18/157,533

(22) Filed: Jan. 20, 2023

Related U.S. Application Data
(60) Provisional application No. 63/301,218, filed on Jan. 20, 2022.

(51) Int. Cl. A61B 34/30; A61B 17/15

(52) U.S. Cl. CPC A61B 34/30; A61B 17/154; A61B 2034/254

(57) ABSTRACT

A robotic cutting guide system for computer-assisted surgery is provided. The robotic cutting guide includes a robotic guide assembly having a cutting guide, a three-dimensional position tracking system for tracking a position of a first bone of a joint, a second bone of the joint, and the robotic guide assembly, and a controller operatively in communication with the robotic guide assembly and the three-dimensional position tracking system. The controller is configured to position the cutting guide adjacent one of the first and second bones of the joint, and then reposition the cutting guide adjacent the other one of the first and second bones of the joint, based on the tracked position of the first and second bones of the joint and the robotic guide assembly.

### BACKGROUND

The present disclosure relates to a robotic cutting guide system for computer-assisted surgery and method for cutting bones. In particular, the present disclosure is related to a computer-assisted surgical system and method for resecting bones of a joint in preparation to receive an orthopedic implant.

Arthroplasty is a surgical procedure to restore the function of a joint, which may be done by either resurfacing the bones or implanting a prosthesis on the joint. In the case of the knee, the prosthesis used during a total knee replacement includes components respectively secured to the distal femur and proximal tibia. Before the prosthesis can be implanted, the bones of the femur and the tibia must be appropriately resected in order to create surfaces for receiving a prosthesis.

To allow for precise bone cuts necessary to ensure accurate prosthesis fit, surgical cutting blocks, or sawing jigs may be used. The jigs may have multiple integrated cutting slots extending at different angles in order to guide saw blades to resect a bone at a fixed predetermined angle. However, depending on the size of the joint, patient anatomy, and the prostheses being used, a different jig may be required for bone resections at different angles outside of the fixed predetermined angles for any particular jig (e.g., within a sagittal plane).

### BRIEF SUMMARY OF THE INVENTION

A robotic cutting guide system for computer-assisted surgery comprising a robotic guide assembly that includes a cutting guide; a three-dimensional position tracking system for tracking a position of a first bone of a joint, a second bone of the joint, and the robotic guide assembly; and a controller operatively in communication with the robotic guide assembly and the three-dimensional position tracking system, wherein the controller is configured to position the cutting guide adjacent one of the first and second bones of the joint, and then reposition the cutting guide adjacent the other one of the first and second bones of the joint, based on the tracked position of the first and/or second bones of the joint and the robotic guide assembly.

The controller is further configured to adjust the position of the cutting guide in six degrees of freedom based on the tracked position of the first and/or second bones of the joint and the robotic guide assembly. The robotic guide assembly further includes a fixation bracket comprising: a primary bracket, and a connection bracket pivotably attached to the primary bracket. The connection bracket further includes secondary pivot having an axis of rotation transverse to an axis of rotation of the primary bracket relative to the connection bracket.

The robotic guide assembly further includes a positioning mechanism having a first arm with a first axis of rotation and a second arm having a second axis of rotation; and a fixation bracket extending from the first arm, and wherein the cutting guide extends from the second arm.

The robotic guide assembly further includes a fixation bracket comprising a primary bracket; and a connection bracket pivotable relative to the primary bracket about a primary axis of rotation, wherein the connection bracket includes pivot having a secondary axis of rotation transverse to the primary axis of rotation, and wherein the primary axis of rotation and the secondary axis of rotation are both transverse to the first axis of rotation of the first arm and the second axis of rotation of the second arm of the positioning mechanism.

The robotic cutting guide system further comprises a joint distraction system for measuring distraction forces between first and second bones of a joint or determining predicted forces and/or predicted gaps between the first and second bones of the joint.

The controller is configured to measure distraction forces between first and second bones of a joint or determine predicted forces and/or predicted gaps between the first and second bones of the joint based on a relative position between the first and second bones of the joint via the joint distraction system. The controller is also configured to determine an optimal position for the cutting guide based on measured distraction forces between first and second bones of a joint or determined predicted forces and/or predicted gaps between the first and second bones of the joint. The controller is also configured to update the position of the cutting guide adjacent the other one of the first and second bones of the joint, based on measured distraction forces between first and second bones of a joint or determined predicted forces and/or predicted gaps between the first and second bones of the joint. The controller is further configured to determine an amount of deviation of the first and second bones from a datum reference and output a warning when the amount of deviation exceeds a predetermined criteria.

The robotic cutting guide system further comprises a pointer, and the controller is configured to display on a display a surgical workflow when the pointer is positioned adjacent a predetermined device. The controller is also configured to change a surgical workflow step when the pointer is positioned adjacent a predetermined device.

A method of resecting bones of a joint for an arthroplasty comprising: using a robotic cutting guide system attaching the robotic guide assembly having the cutting guide to a first bone of a joint; tracking a position of the first bone of the joint, a second bone of the joint, and the robotic guide assembly; positioning the cutting guide adjacent the first bone based on the tracked position of the first bone of the joint and the robotic guide assembly for resecting the first bone; and repositioning the cutting guide adjacent the second bone based on the tracked position of the first and second bones of the joint and the robotic guide assembly for resecting the second bone. Repositioning of the cutting guide adjacent the second bone is completed while the robotic guide assembly is attached to the first bone.

The method further comprises adjusting the position of the cutting guide relative to one of the first and second bones of the joint up to six degrees of freedom. The method further comprises, using a joint distraction system, measuring distraction forces between first and second bones of the joint or determining predicted forces and/or predicted gaps between the first and second bones of the joint based on a relative position between the first and second bones of the joint. The method further comprises, using a joint distraction system, measuring distraction forces between first and second bones of the joint or determining predicted forces and/or predicted gaps between the first and second bones of the joint based on a relative position between the first and second bones of the joint after an implant has been implanted to one of the first and second bones of the joint. The method further comprises, using a joint distraction system, measuring distraction forces between first and second bones of the joint or determining predicted forces and/or predicted gaps between the first and second bones of the joint based on a relative position between the first and second bones of the joint after resecting bone on only one of the first and second bones of the joint.

The subject disclosure further provides a method of resecting bones of a joint for an arthroplasty. The method includes attaching a robotic guide assembly having a cutting guide to a first bone of a joint, and using a robotic cutting guide system, tracking a position of the first bone of the joint, a second bone of the joint, and the robotic guide assembly. The method further includes, using a robotic cutting guide system, positioning the cutting guide adjacent the first bone based on the tracked position of the first and second bones of the joint and the robotic guide assembly for resecting the first bone, and repositioning the cutting guide adjacent the second bone based on the tracked position of the first and second bones of the joint and the robotic guide assembly for resecting the second bone. The method further includes, using a robotic cutting guide system, adjusting the position of the cutting guide relative to one of the first and second bones of the joint up to six degrees of freedom. The method further includes changing a step within a surgical workflow being performed by the robotic cutting guide system by moving a pointer tracked by the three-dimensional position tracking system to a predetermined position adjacent a predetermined device.

### BRIEF DESCRIPTION OF THE SEVERAL VIEWS OF THE DRAWINGS

The foregoing summary, as well as the following detailed description of the subject disclosure, will be better understood when read in conjunction with the appended drawings. For the purpose of illustrating the subject disclosure, there are shown in the drawings exemplary embodiments of the invention. It should be understood, however, that the exemplary embodiments are not limited to the precise arrangements and instrumentalities shown.

In the drawings:

FIG. 1 is a perspective view of a robotic cutting guide system in accordance with an exemplary embodiment of the subject disclosure;

FIG. 2 is a perspective view of a robotic guide assembly of the robotic cutting guide system of FIG. 1 attached to a femur of a knee joint;

FIG. 3 is another perspective view the robotic guide assembly of FIG. 2;

FIG. 4 is an anterior perspective view of the robotic guide assembly of FIG. 2;

FIG. 5 is a medial side view of the robotic guide assembly of FIG. 2;

FIG. 6 is a perspective view of various anatomical planes of a knee joint;

FIG. 7 is a perspective view of an exemplary joint distraction device applicable to the robotic cutting guide system of FIG. 1;

FIG. 8 is side view of an exemplary surgical leg holder applicable to the robotic cutting guide system of the subject disclosure;

FIG. 9 is a perspective view of an exemplary marker in accordance with another exemplary embodiment of the subject disclosure; and

FIGS. 10 and 11 are schematic surgical workflow diagrams applicable to the exemplary embodiments of the subject disclosure.

### DETAILED DESCRIPTION OF THE INVENTION

Reference will now be made in detail to the various exemplary embodiments of the subject disclosure illustrated in the accompanying drawings. Wherever possible, the same or like reference numbers will be used throughout the drawings to refer to the same or like features. It should be noted that the drawings are in simplified form and are not drawn to precise scale. Certain terminology is used in the following description for convenience only and is not limiting. Directional terms such as top, bottom, left, right, above, below and diagonal, are used with respect to the accompanying drawings. The term “distal” shall mean away from the intermediate of a body. The term “proximal” shall mean closer towards the intermediate of a body and/or away from the “distal” end. The words “inwardly” and “outwardly” refer to directions toward and away from, respectively, the geometric intermediate of the identified element and designated parts thereof. Such directional terms used in conjunction with the following description of the drawings should not be construed to limit the scope of the subject application in any manner not explicitly set forth. Additionally, the term “a,” as used in the specification, means “at least one.” The terminology includes the words above specifically mentioned, derivatives thereof, and words of similar import.

“About” as used herein when referring to a measurable value such as an amount, a temporal duration, and the like, is meant to encompass variations of ±20%, ±10%, ±5%, ±1%, and ±0.1% from the specified value, as such variations are appropriate.

“Substantially” as used herein shall mean considerable in extent, largely but not wholly that which is specified, or an appropriate variation therefrom as is acceptable within the field of art. “Exemplary” as used herein shall mean serving as an example.

Throughout the subject application, various aspects thereof can be presented in a range format. It should be understood that the description in range format is merely for convenience and brevity and should not be construed as an inflexible limitation on the scope of the subject disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Furthermore, the described features, advantages and characteristics of the embodiments of the invention may be combined in any suitable manner in one or more embodiments. One skilled in the relevant art will recognize, in light of the description herein, that the invention can be practiced without one or more of the specific features or advantages of a particular embodiment. In other instances, additional features and advantages may be recognized in certain exemplary embodiments that may not be present in all embodiments of the subject disclosure.

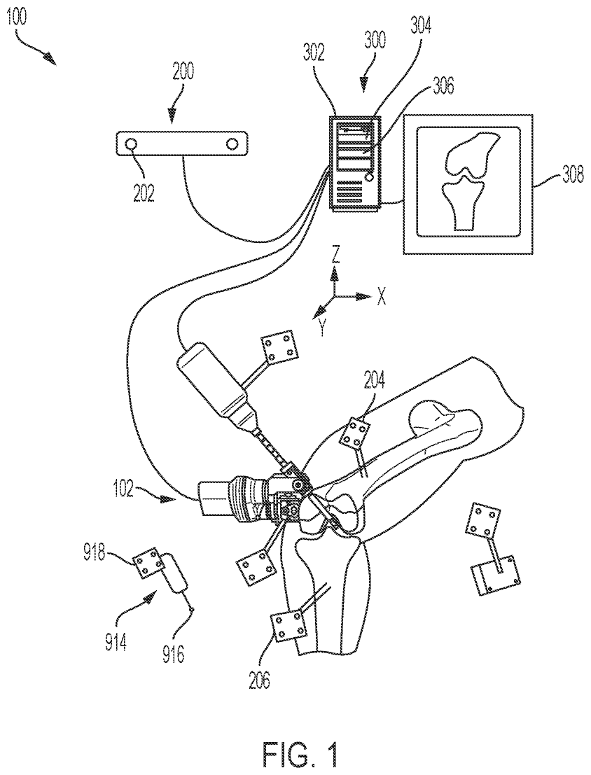

Referring to FIG. 1, the subject disclosure provides a robotic cutting guide system 100 for computer-assisted surgery, such as e.g., orthopedic arthroplasty surgery. The robotic cutting guide system 100 includes a robotic guide assembly 102, a three-dimensional position tracking system 200 for tracking a position of a joint and other bones of a patient and the robotic guide assembly, and a controller 300 operatively in communication with the robotic guide assembly and the three-dimensional position tracking system.

In accordance with an exemplary embodiment of the subject disclosure, the robotic guide assembly 102 is configured as shown in FIGS. 1-5. The robotic guide assembly 102 includes a positioning mechanism 108, a fixation bracket 110, and a cutting guide 112. The robotic guide assembly is also operatively in communication with the controller 300 via a wired 111 or wireless 114 connection.

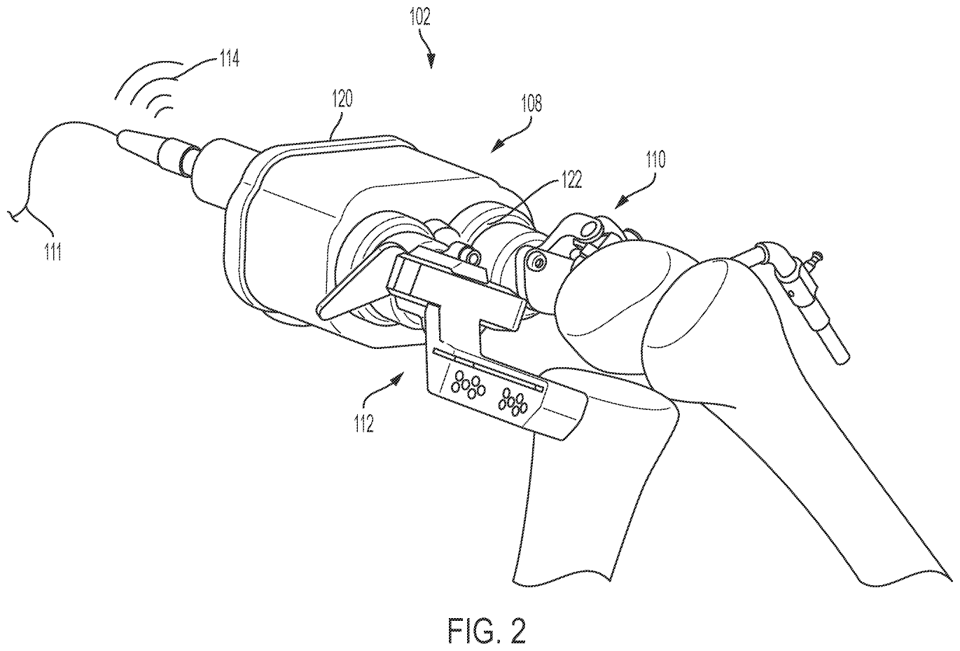

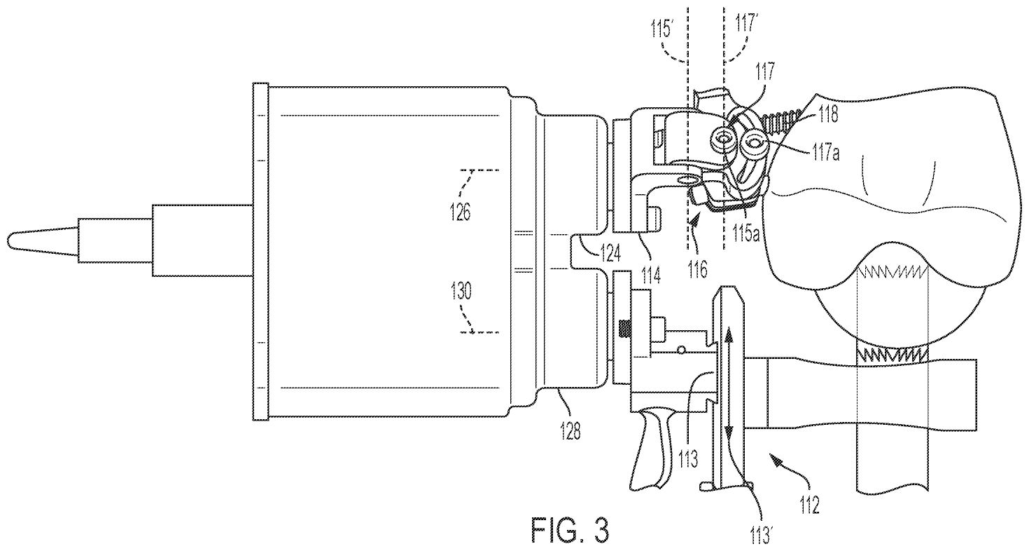

The fixation bracket 110 is configured as best shown in FIGS. 2 and 3. The fixation bracket is configured to attach the robotic guide assembly to a medial or lateral side of a bone. For example, the robotic guide assembly can be attached to a medial or lateral side of a distal femur of a knee joint. While the foregoing description of the subject disclosure will be in reference to an exemplary knee joint, the subject disclosure can be applied to any bone of a patient or any joint of a patient including, but not limited to, a hip joint, a shoulder joint, an elbow joint, an ankle joint, and the like.

The fixation bracket 110 includes a primary bracket 114 connected to the positioning mechanism 108, and a connection bracket 116 configured to receive fasteners 118 for fastening the fixation bracket to bone and thereby the robotic guide assembly. The fixation bracket is pivotably connected to the connection bracket 116 via a pivot 115 having a longitudinal axis 115′ about which the fixation bracket pivots about. The longitudinal axis 115′ of the pivot 115 is transverse to a rotational axis of a first or second axis of rotation of the positioning mechanism, as further discussed below. When attached to a lateral side of a distal end of a femur, the axis 115′ is aligned with a transverse plane of the femur (i.e., that axis of rotation for pivot 115 is parallel to a normal vector to the transverse plane). In accordance with an aspect of the subject disclosure, the longitudinal axis of the pivot is about 90 degrees relative to the first or second axis of rotation of the positioning mechanism. This rotational freedom provided by the pivot 115 allows the robotic guide assembly to move and adjust the position of the cutting guide in the varus/valgus direction. The pivot 115 is fastened or secured in position by a fastener 115a, e.g., a screw, which locks the position of the pivot about its pivot axis.

The connection bracket 116 also includes a secondary pivot 117 which pivots about its pivot axis 117′. The pivot axis 117′ is transverse to the longitudinal axis 115′ of pivot 115 and when attached to a lateral side of the distal femur, the pivot axis 117′ is aligned with a frontal plane of the femur. The pivot 117 is fastened or secured in position by a fastener 117a, e.g., a screw, which locks the position of the pivot about its pivot axis.

In accordance with another exemplary embodiment, a motor can be operatively connected to the pivot 115 to move the positioning mechanism about the pivot. A motor can also be operatively connected to the pivot 117 to move the positioning mechanism and cutting guide about the pivot. These motors can be controlled by the controller 300 to provide automatic positioning of the pivot 115 or 117 and thereby the robotic guide assembly e.g., based on the tracked position of a bone or bones, the positioning mechanism, the motor, the cutting guide or any other instrument or marker tracked by the three-dimensional position tracking system. Additional axes can also be incorporated and can be motorized and controlled to provide additional degrees of freedom and range of movement to the cutting guide and robotic guide assembly.

The positioning mechanism can be a positioning motor. In accordance with an exemplary embodiment, the positioning mechanism 108 can be configured as shown in FIGS. 2 and 3. The positioning mechanism 108 includes a body 120, a motor 122, a first section or first arm 124 having a first axis of rotation 126, and a second section or second arm 128 having a second axis of rotation 130. The first and second arms are spaced from each other and independently rotatable about their respective axes.

The motor 122 is operatively connected to each of the first and second arms for driving rotation of each arm respectively about their respective axes. Alternatively, the robotic guide assembly can include first and second motors each for respectively driving rotation of the first and second arms. As such, the robotic guide assembly is constructed to have at least two rotational degrees of freedom.

The first arm 124 has a medial end connected to the fixation bracket. The second arm 128 has a medial end connected to the cutting guide 112. As such, the first arm can rotate relative to the fixation bracket and the second arm can rotate the cutting guide relative to the body 120.

The cutting guide 112 is an exemplary cutting guide applicable to the subject disclosure and configured as best shown in FIGS. 2 and 3. The cutting guide can alternatively be configured as any other cutting guide suitable for its intended purposes, such as an open face cutting guide, a captured cutting slot, a drill guide, a milling or burring tool guide, and the like.

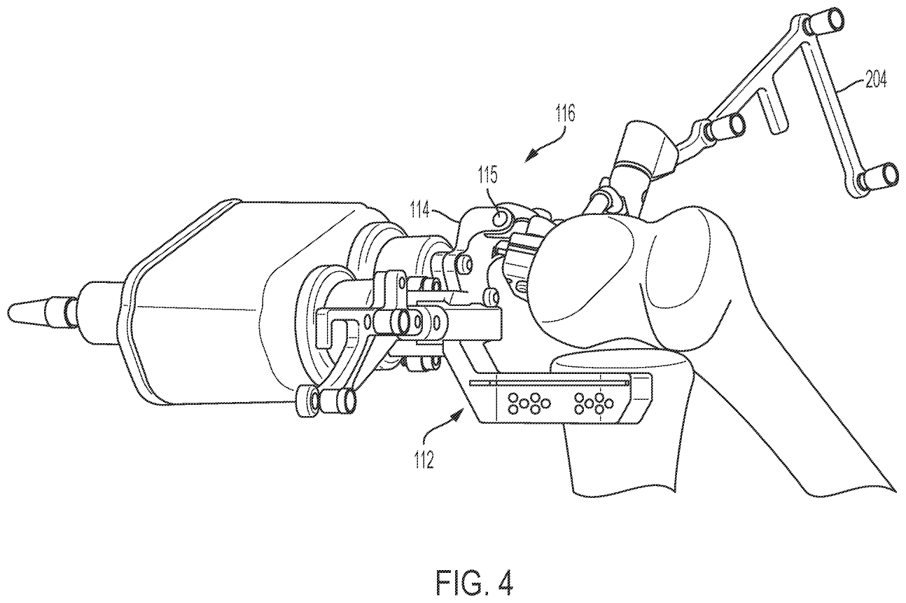

Referring to FIG. 4, the cutting guide 112 can optionally include one or more through holes configured to receive a pin for releasably fixating the cutting guide to a bone.

Various features of the positioning mechanism applicable to the subject disclosure can be configured as disclosed in U.S. Pat. Nos. 9,421,019 and 10,383,638, the entire disclosures of which are hereby incorporated by reference in their entirety for all purposes. The robotic guide can have two, three, four, five, six or seven motorized degrees of freedom and can be made up of a series of rotational actuators, one per degree of freedom, as in a robotic arm. It can also have linear actuators for sliding a segment or number of segments of the robotic cutting guide in a linear direction. For instance, the cutting guide 112 can slide towards and away from the bone in a linear motion along direction 113′ using guiderail 113. This motion can either be manually controlled by the operator using a mechanical locking and unlocking mechanism, or the motion can be controlled automatically by a motor operatively connected to the cutting guide and the controller.

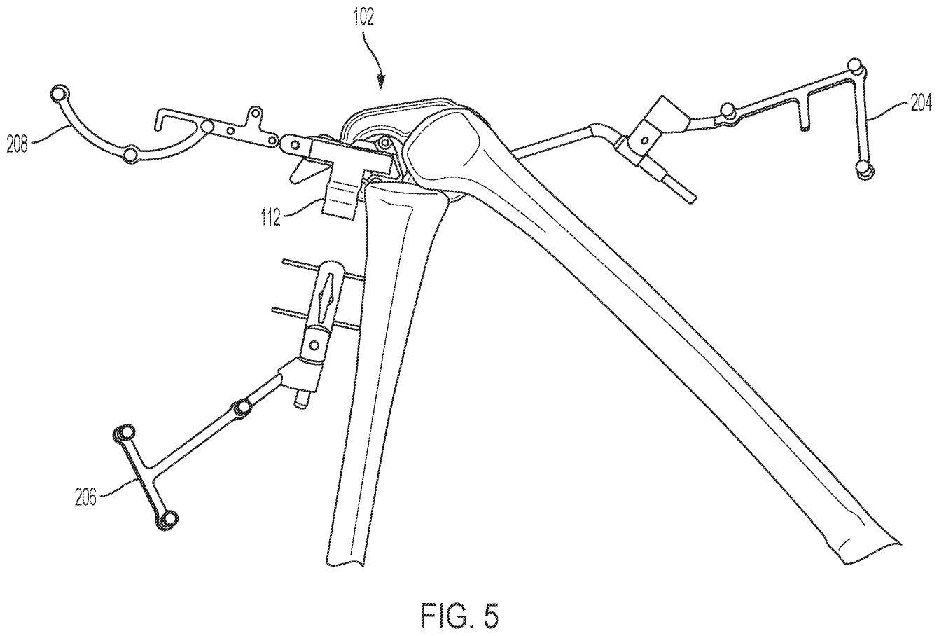

In accordance with an exemplary embodiment of the subject disclosure, the three-dimensional position tracking system 200 includes a position measuring device 202 capable of tracking the positions of markers in three-dimensional space in real time. The markers 204, 206 and 208 (FIG. 5) can be attached e.g., to a first bone of a joint such as a femur, a second bone of a joint such as a tibia, and the robotic guide assembly 102. As such, the tracking system 200 can track the position of the first and second bones of the joint throughout a range of motion in real time relative to the position of the robotic guide assembly.

Various features of a three-dimensional position tracking system applicable to the subject disclosure are disclosed in U.S. Pat. Nos. 9,421,019 and 10,383,638, the entire disclosures of which are hereby incorporated by reference in their entirety for all purposes.

The controller 300 is operatively in communication with the robotic guide assembly and the three-dimensional position tracking system e.g., via a wired or wireless connection. The controller can be a computer having a memory and a processor for executing computer instructions stored in the memory, or a processor, or logic controller, and the like. In sum, the controller 300 is configured to position the cutting guide adjacent one of the first and second bones of the joint, and then reposition the cutting guide adjacent the other one of the first and second bones of the joint, based on the tracked positions of the first and second bones of the joint and the robotic guide assembly.

In accordance with an exemplary embodiment, the controller 300 is a computer 302 having a memory 304 and a processor 306. The memory 304 includes computer instructions executable by the processor to operate the robotic guide assembly 102. The memory 304 can be configured to store pre-determined cut configurations of the first and second bones to receive a specific type of orthopedic implant or brand of orthopedic implant.

The memory also includes instructions executable such that the processor configures an optimal position for the cutting guide based on the position of the first bone, the second bone, and the robotic guide assembly to allow a user to make the pre-determined cut configurations of the first and second bones. That is, the controller adjusts the position of the robotic guide assembly and/or the cutting guide based on the position of the first bone, the second bone, and the robotic guide assembly, such as a flexion angle of the joint when the joint moves about its range of motion, internal/external rotation of one of the first and second bones, or in a varus/valgus direction of motion.

In accordance with another exemplary embodiment, the robotic guide assembly 102 can be attached to the first bone and positioned relative to the second bone using the tracked position of the second bone and the tracked position of the robotic guide assembly, without the need for tracking the position of the first bone. In other words, it is not necessary to track the position of the first bone when the robotic guide assembly is being positioned relative to the second bone, for example to position the cutting guide of the robotic guide assembly adjacent the tibia for making a resection on the tibia, when the robotic guide assembly is fixed to the femur via the fixation bracket. Since the position of the robotic guide assembly (and consequently the cutting guide) and the second bone are tracked, there is no need for tracking the position of the first bone to which the robotic guide assembly is attached to when positioning the cutting guide for a tibial resection. The robotic guide assembly can be calibrated in space relative to either the first bone marker, the cutting guide marker (or robotic guide assembly marker), or the second bone marker. As such, the robot guide assembly can also position itself relative to an adjacent bone using only two tracking arrays or markers. The use of only two tracking arrays or markers reduces the field of view required of the three-dimensional position tracking system during cutting guide positioning and allows for more personnel in the operating room to stand around the operative site without causing issues associated with obstructing the line of site to the arrays/three-dimensional position tracking system.

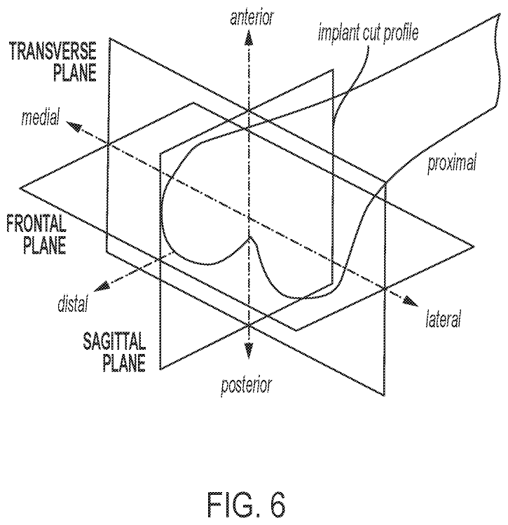

Due to the configuration of the robotic guide assembly including its fixation bracket, and first and second arms, the controller 300 can adjust the position of the cutting guide in six degrees of freedom based on the tracked positions of the first bone, the second bone and the robotic guide assembly. That is, the controller can move the cutting guide or angle the cutting guide about a sagittal plane, a transverse plane, or a frontal plane of one of the first and second bones of the joint (FIG. 6).

Additionally, the controller is configured to track the position of the first and/or second bones and the robotic guide assembly via the three-dimensional position tracking system, and to adjust or reposition the robotic guide assembly in real time. That is, the robotic guide assembly adjusts its position in real time based on the real time tracked positions of the first and/or second bones and the robotic guide assembly. For example, if the robotic guide assembly is attached to a first bone e.g., a femur, and is positioning its cutting guide relative to a second bone, e.g., a tibia, once the cutting guide is positioned to a predetermined desired position to resect the tibia and subsequent motion of the first and/or second bones occurs or subsequent motion of the second bone relative to the first bone and/or the robotic guide assembly occurs, the robotic guide assembly simultaneously adjusts or repositions itself to correct for said motions such that the cutting guide of the robotic guide assembly remains aligned in the predetermined desired position. In other words, the robotic cutting guide assembly can compensate for motions of one or more bones of the joint e.g., motions of the tibia relative to femur, by moving, adjusting, or repositioning the robotic guide assembly in real time as the motions of the one or more bones of the joint.

Various features of a controller applicable to the subject disclosure are disclosed in U.S. Pat. Nos. 9,421,019 and 10,383,638, the entire disclosures of which are hereby incorporated by reference in their entirety for all purposes.

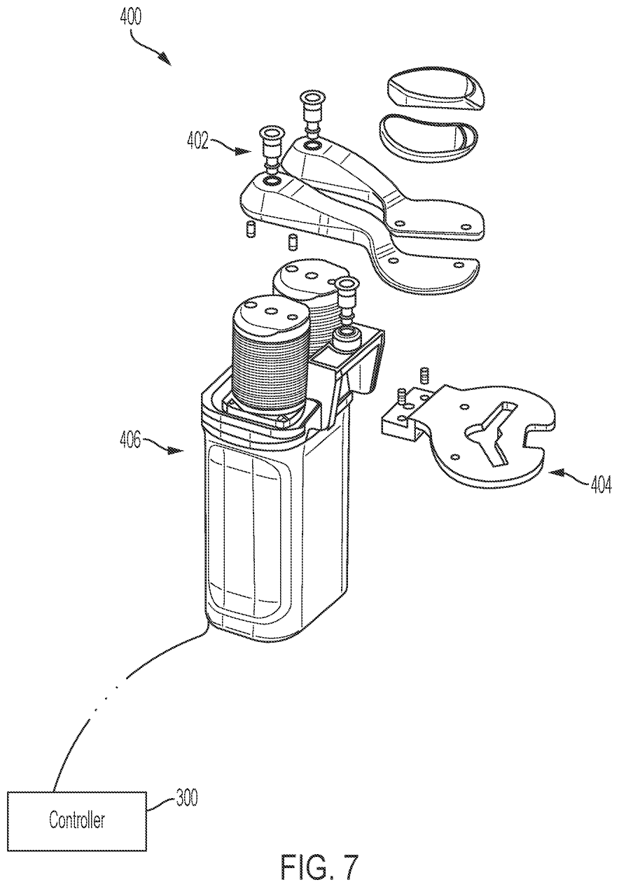

In accordance with another exemplary embodiment of the subject disclosure, the robotic cutting guide system 100 can further include a joint distraction system 400 (FIG. 7). The joint distraction system includes an upper paddle 402 for engaging a first bone of a joint, a lower paddle 404 for engaging a second bone of the joint, and a displacement mechanism 406 operable to displace the upper paddle relative to the lower paddle. The joint distraction device is operatively connect to and in communication with the controller 300. The controller 300 can be configured to apply varying displacement forces to displace the upper paddle from the lower paddle based on a relative position between the first and second bones of the joint.

The controller measures distraction forces between the first and second bones of the joint and determines predicted forces and/or predicted gaps between the bones of the joint e.g., based on the distraction forces. The determined predicted forces and/or predicted gaps are communicated to the controller, which then determines an optimal position for the cutting guide based on the positions of the first bone, the second bone, the robotic guide assembly, and the determined predicted forces and/or predicted gaps between the bones of the joint from the joint distraction system.

Various features of a joint distraction device applicable to the subject disclosure are disclosed in U.S. Pat. No. 10,321,904 the entire disclosure of which is hereby incorporated by reference in its entirety for all purposes.

When the joint distraction system 400 is used in combination with the robotic guide assembly 102, the controller is configured to compensate for the additional forces imparted on the joint due to the weight of the robotic guide assembly. For example, when the robotic guide assembly is attached to a medial side of the distal femur, the joint distraction system applies corresponding counterforces or compensating forces to the medial condyle of the femur to zero out the forces imparted by the weight of the robotic guide assembly. Similarly, when the robotic guide assembly is attached to a lateral side of the distal femur, the joint distraction system applies corresponding counterforces to the lateral condyle of the femur to zero out the forces imparted by the weight of the robotic guide assembly.

Additionally, the joint distraction system 400 can be used to assess the knee joint after a tibial resection cut has been made using the robotic guide assembly 102. In particular, after the tibia has been resected and/or trial tibial implants inserted, the joint distraction system can be inserted to assess the joint forces and balance of the knee while the robotic guide assembly remains attached to the femur. In other words, the joint distraction system can be applied at any time the robotic guide assembly is concurrently attached to the femur.

The following is an exemplary example of the robotic cutting guide system 100 used in a total knee arthroplasty. A femoral bone marker 204 in the shape of a letter F is attached to a femur, e.g., about the distal femur of a knee joint, a tibial bone marker 206 in the shape of a letter T is attached to a tibia of the knee joint about the proximal tibia, and a guide marker 208 in the shape of a letter G is attached to the robotic guide assembly. After appropriate registrations of the femoral bone, the tibial bone and the guide marker, the three-dimensional position tracking system 200 is then used to track the relative positions of the tibia, femur and robotic guide assembly during surgery or intra-operatively. As shown in FIG. 4, the robotic guide assembly 102 is attached to the lateral side of the distal femur, e.g., in proximity to the epicondyles. The robotic guide assembly is attached to the femur e.g., via bone screws and/or bone pins.

After attachment of the robotic guide assembly to the femur, the body 120 of the robotic guide assembly can rotate about the first axis of rotation 126 due to operation of the first arm 124. The second arm 128 can also rotate about its second axis of rotation to rotate the position of the cutting guide 112 attached to a medial end thereof.

Based on the position of the femur, the tibia, and the robotic guide assembly, the controller positions the cutting guide to a position about the distal femur corresponding to an applicable pre-determined cut or cut configuration of the subject femoral implant to be implanted on the femur. After all femoral bone cuts are made, the controller repositions the cutting guide to a position about the proximal tibia corresponding to an applicable pre-determined cut or cut configuration of the subject tibial implant to be implanted on the tibia. The cutting guide is repositioned to resect the tibia while the robotic guide assembly remains affixed to the distal femur.

The positioning of the cutting guide adjacent the femur or the tibia can be further adjusted about the sagittal plane via movement about the pivot 115. This adjustment can be controlled by the controller or manually adjusted via manual rotation of the body 120 about the pivot 115. The pivot 115 also allows for pivotable movement about the transverse plane of the femur and the frontal plane of tibia (when the knee is flexed to about 90 degrees of flexion as shown in FIG. 3).

Optionally, the joint distraction system 400 can be used to determine predicted forces and/or predicted gaps between the femur and tibia. The controller can then determine the position of the cutting guide adjacent the femur and tibia based on the positions of the femur, the tibia, and the robotic guide assembly, and the determined predicted forces and/or predicted gaps between the femur and tibia from the joint distraction system.

The robotic cutting guide system of the subject disclosure advantageously provides for a single universal tool for all planar bone cuts of a joint e.g., on the femur and tibia of a knee joint, an automatic tibial cut-guide positioning for posterior slope, flexion, and cutting height, adjustable positioning for varus/valgus, and simultaneous adjustment in all degrees of freedom. The robotic cutting guide system also advantageously provides for a tibial cutting guide that does not require pins for securing the guide to the tibia which can save time and reduces invasiveness and complexity. The foregoing system also allow for compatibility with anterior or medial saw approaches.

The robotic cutting guide system of the subject disclosure can be used to resect bones of first and second bones of a joint to accept a respective implant. The robotic cutting guide system can be configured to cut a first bone e.g., a femur of a knee joint, first followed by resecting a second bone e.g., a tibia of a knee joint. Alternatively, the second bone can be resected first and then the first bone can be resected subsequent to the second bone. Additionally, the joint distraction device can be used to (e.g., as discussed above) to measure forces and determine predicted forces and/or predicted gaps before any bone cuts of the bones of a joint, after a first bone of a joint is resected and before any bone cuts on the second bone of a joint, or after all or most of the bone cuts of the first and second bones of the joint are made.

Additionally, the robotic cutting guide system and/or joint distraction device can be used to make the appropriate bone resections of a joint after one of the first and second bones of the joint have been implanted with a respective implant. For example, in connection with a knee joint, the joint distraction device can be configured to measure forces and determine predicted forces and/or predicted gaps after a femoral implant has been implanted to the femur and a femoral implant registration has been completed to allow the robotic cutting guide system to track the femoral implant. Alternatively, the joint distraction device can be configured to measure forces and determine predicted forces and/or predicted gaps after a tibial implant has been implanted to the tibial bone and a tibial implant registration has been completed.

After the joint distraction device measures the forces and/or determines predicted forces and/or predicted gaps between the bones of the joint (before or after implantation of implant(s)), the controller is configured to receive said data and determine if said measured forces and/or determined predicted forces and/or predicted gaps match or meets a criteria established by a predetermined surgical plan. If said data does not match or meet the criteria of the surgical plan, the controller is then configured to adjust or update the robotic guide assembly to reposition the cutting guide in an updated position to provide a best match position for meeting the criteria established by the surgical plan.

In accordance with another exemplary embodiment, the subject disclosure provides a method of monitoring and/or alerting a user to movement of the robotic cutting guide with respect to bone (e.g., one or more bones) that exceeds a predetermined threshold. The robotic cutting guide system monitors the position and movement of the robotic cutting guide and/or the motion of the first and second bones of a joint that is the subject of the surgery. The first and second bones of the joint are tracked with the three-dimensional position tracking system 200 throughout the duration of the subject surgery, including during the positioning the cutting guide 112 relative to the first and second bones, and during the resection using the cutting guide. If there is motion above a specific threshold during the resection using the cutting guide, the surgeon can be alerted that the cutting guide has deviated from the targeted position and can stop the cutting process to correct the position of the cutting guide of the robotic guide assembly or the bone or to apply less force on the cutting guide to prevent the cutting guide from deviating from a target.

In this exemplary embodiment, the controller's memory 304 has stored thereon a predetermined criteria or threshold or deviation for acceptable movement or positioning of the first and second bones of the joint from its sagittal plane, or movement of the first and second bones from a plane of the cutting guide 112, or some other datum reference. The predetermined criteria can be e.g., +/−0.1, 0.2, 0.5, 1.0, 1.5, 2.0, 2.5, 3.0, 3.5, 4.0, 5.0, 6.0, 7.0, 8.0, 9.0 or 10 degree or +/1 0.1, 0.2, 0.5, 1.0, 1.5, 2.0, 2.5, 3.0, 3.5, 4.0, 5.0, 6.0, 7.0, 8.0, 9.0 or 10 mm.

The controller 300 is configured to determine an amount of deviation of the first and second bones from a datum reference e.g., a sagittal plane of the first and second bones, or a plane of the cutting guide, based upon a tracking of the first and second bones. The controller is then configured to output a warning or indicator or signal to a user when the amount of deviation of the first and second bones from the datum reference exceeds the predetermined criteria or threshold or deviation. The output can be displayed on a display as a visual warning or an audible output as an audible warning, or a physical output such as a vibration of a part or feature of the robotic cutting system as a physical warning. In other words, the controller has stored in memory computer instructions executable by the processor to perform the above functions of the controller, e.g., warning function, indicator function or signaling function.

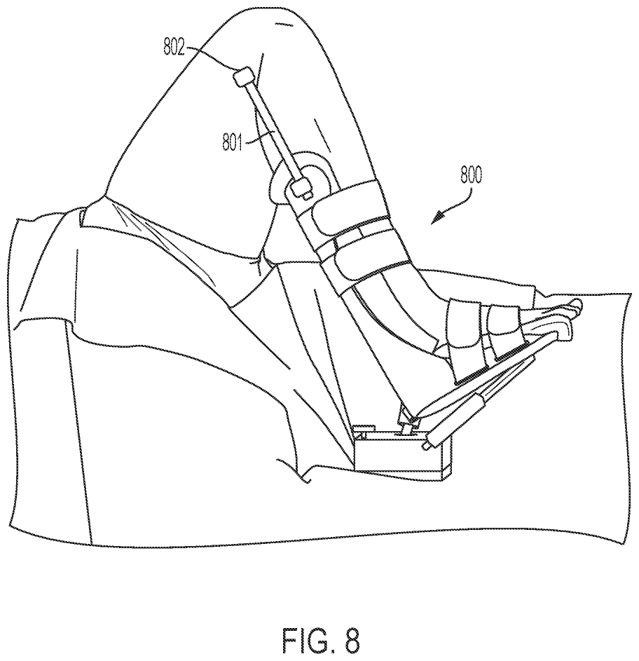

In accordance with another exemplary embodiment, the subject disclosure provides a surgical leg holder 800, a best shown in FIG. 8. The surgical leg holder secures a patient's bone e.g., a femur or tibia, in place to stabilize the joint during the subject surgery using the robotic cutting guide system. The leg holder can also be used to attach the robotic cutting guide to, instead of attaching it to the bone as previously described. This makes the robotic cutting guide system less invasive as it reduces the size of the skin and muscle incision for attachment of the robotic guide assembly to bone, thereby permitting a less invasive surgery.

The leg holder can also be configured with a support member 801 having a far end 802 for attaching the robotic guide assembly thereto such that the cutting guide 112 is in the vicinity of the joint to be operated on and the joint is within a workspace and range of movement of the cutting guide. The support member can be adjustable and can have one or more articulations to position the far end 802 close to the operation site and accommodate a range of patient anatomies and limb lengths. The attachment of the robotic guide assembly to the support member and of the support to the leg holder can be performed with quick release attachment and locking mechanisms to allow for quick and easy attachment and detachment of the robotic guide assembly. This allows for the robotic guide assembly to be brought into the surgical field when required and removed afterwards so as not to inhibit the surgery or take up space around the joint when not in use.

In accordance with a further exemplary embodiment, the robotic cutting guide system is configured to allow a user to access surgical workflows intraoperatively to move between various steps within a given surgical workflow. In this exemplary embodiment, the memory 304 of the controller includes computer instructions executable by the processor for carrying out various surgical workflows. That is, one or more of the various surgical workflows stored in memory is pre-selected by the user for a particular surgical procedure to be carried out by the robotic cutting guide system.

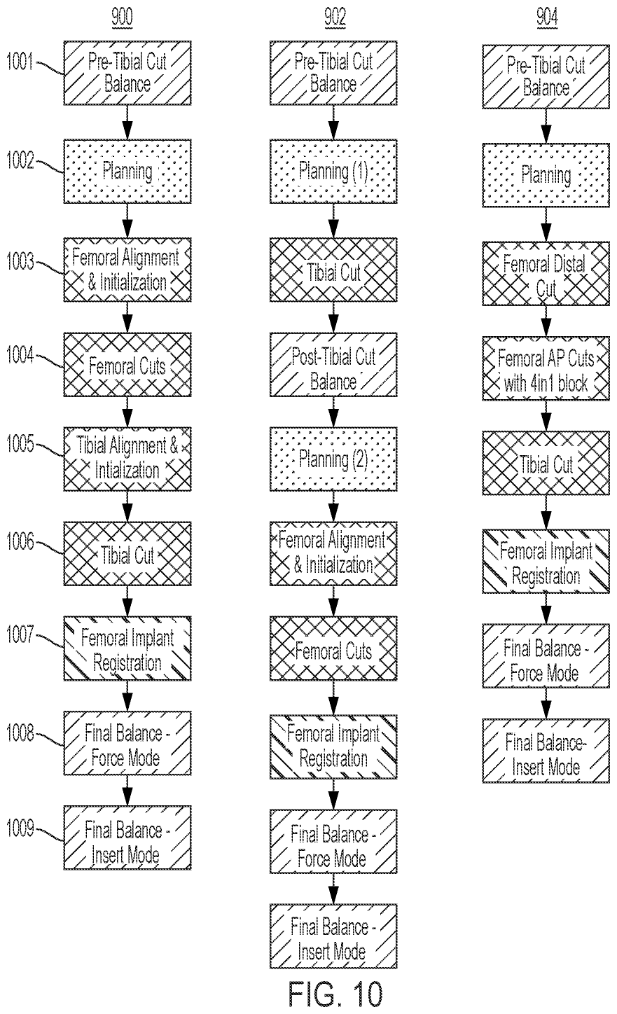

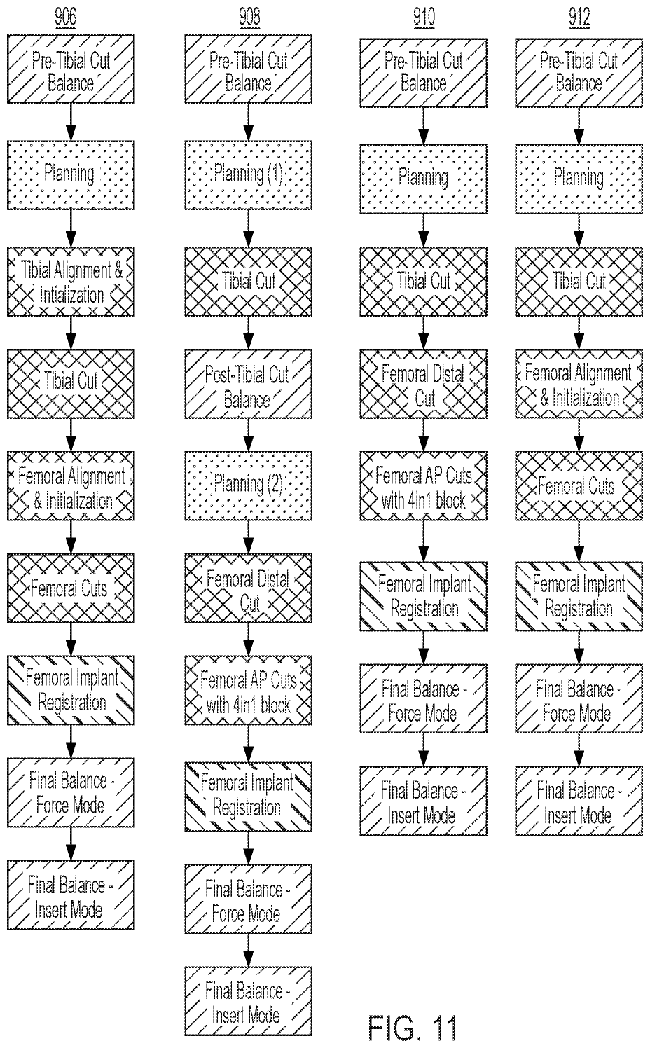

Exemplary surgical workflows can include workflows 900, 902, 904, 906, 908, 910, and 912, as illustrated in FIGS. 10 and 11. Workflow 900 includes but is not limited to the following steps: pre-tibial resection balancing 1001, surgical planning 1002, femoral alignment and initialization of the femur 1003, femoral resection of the femur 1004, trial alignment and initialization 1005, tibial resection 1006, femoral implant registration 1007, final joint balancing via force mode with a joint distraction system 1008, and final joint balancing via insert mode with a joint distraction system 1009.

More specifically, the workflow 900 can include the initial steps of instrument calibration, initial bone anatomy registration and limb alignment assessment, followed by pre-tibial resection balance 1001, where the upper paddle or paddles of the joint distraction system are inserted into the joint prior to resection of the tibia and the femur and pre-resection gap data is collected as the knee is taken through a range of flexion while a controlled load is being applied to the joint by the controller and joint distraction system. Planning 1002 can then include combined planning of the tibia and femoral cuts and implants with medial and/or lateral predicted gap curves that are displayed at various flexion angles or throughout the range of motion as predicted gap profiles, based on the pre-tibial cut balance acquisition data. The robotic guide assembly can then be attached to the femur and aligned with the three-dimensional position tracking system and initialized (calibrated) relative to the femur. Femoral cuts 1004 can then be performed using the robotic guide assembly. The robotic guide assembly can then be aligned and initialized and positioned relative to the tibia (step 1005). The tibia can then be resected using the robotic guide assembly (step 1006). The implants and/or the bone cut surfaces can be registered to record where the implants have been positioned (1007). A joint distraction system can then be reinserted into the joint with the femoral trial or final implant in place and a final balance acquisition can be made under controlled loading to record and display the final gaps, and an insert mode acquisition can be made were the joint distraction device replicates the final tibial insert thickness and records and displays the loads between the joint distraction device, which is acting as the tibial insert, and the femoral implant or trial implant. While the foregoing surgical workflows are illustrative of a total knee replacement procedure, the surgical workflows can alternatively include workflows for other joints and other applicable joint procedures, such as, e.g., partial or unicondylar knee arthroplasty, the hip, elbow, shoulder and ankle. The robotic cutting guide and described methods of use could also be adapted to support both total and partial arthroplasty procedures in the above-mentioned joints.

This exemplary embodiment also includes a pointer 914 having a ball tip 916 and a tracking array 918 for tracking the position of the pointer with the three-dimensional position tracking system 200. The pointer 914 is configured to register the anatomy of the bones of the joint. The ball tip 916 can be a ball tip having a 1 mm radius or alternatively the ball tip can be a pointer tip having any other suitable shape and/or configuration, e.g., a pointed tip. The pointer is calibrated with respect to the tracking array 918 and therefore can be tracked in 3D space and relative to other arrays via the three-dimensional position tracking system.

In accordance with an aspect of the subject disclosure, the controller is configured to display on a display a surgical workflow when the pointer is positioned adjacent a predetermined device, e.g., a marker. That is, the controller is configured to determine the position of the pointer 914 relative to e.g., marker 204 and 206. Once a predetermined proximity of the pointer relative to the marker 204 or 206 is detected, the controller can output the specific workflow step the system is performing e.g., on a display 308. Additionally, depending on which marker the pointer is determined to be in proximity to, the controller can move the system forwards or backwards within the workflow being performed by the system. That is, the user can guide the system to move forwards or backwards within the instant workflow by bringing the pointer towards the marker 204 e.g., to move the workflow forwards, or by bringing the pointer towards the marker 206 e.g., to move the workflow backwards. The proximity within which the user is required to bring the pointer to the respective marker can be any reasonable proximity, e.g., 50, 40, 30, 20, 10, or 5 mm. In other words, the controller is configured to change a surgical workflow step when the pointer is positioned adjacent a predetermined device, e.g., a predetermined device.

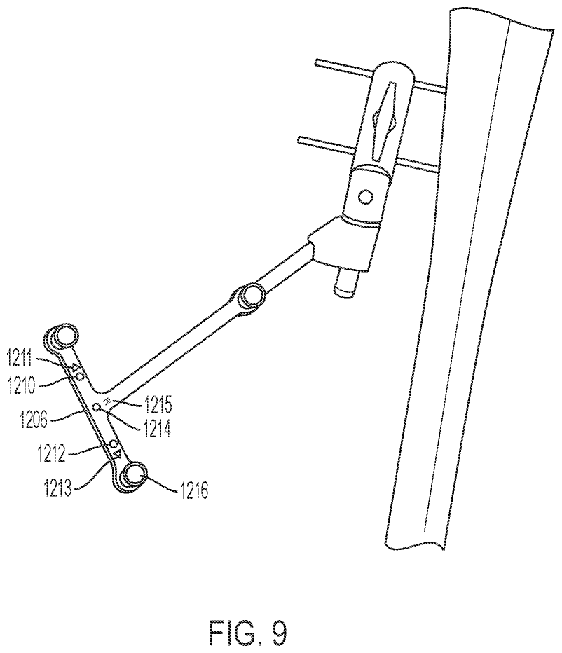

Alternatively, in accordance with another aspect and referring to FIG. 9, one or more of the markers 1206 can have markings 1211, 1213 and corresponding dimples or cones 1210, 1212 for receiving the pointer tip. The position of the cones is known relative to the coordinate system of the marker 1206. The cones can also have markings adjacent to it to identify it, such as, “forward,” or a forward arrow, or a “>” symbol 1211, and the second cone can be labeled “back,” or have a backward arrow, or a “<” symbol 1213. Then, when the pointer tip is inserted into a specific cone, the three-dimensional position tracking system can measure the position of the probe tip with respect to the marker 1206, and because the position of the cones are known with respect to the marker, the controller can determine and detect that the position of the tip is close to or inside one of the cones and will automatically control the system to go to a prior step or subsequent step in the instant surgical workflow depending on which cone is selected.

Additionally, the marker 1206 can be configured to include a marking 1215 e.g., “M” and a middle cone 1214, that accesses and brings up a menu on the display of each step in the instant surgical workflow. Thereafter, the user can use the forward or backward cones to navigate within the menu up or down in the surgical workflow. That is, the controller can be configured to allow the user to move multiple steps forward or backward within the instant surgical workflow by repeatedly brining the pointer tip in and out of a respective cone. The middle cone or main menu cone can then be used to select or validate a workflow step selection.

It will be appreciated by those skilled in the art that changes could be made to the exemplary embodiments described above without departing from the broad inventive concept thereof. It is to be understood, therefore, that the subject disclosure is not limited to the particular embodiments disclosed, but it is intended to cover modifications within the spirit and scope of the present invention as discussed above and claimed.

I/We claim:

1. A robotic cutting guide system for computer-assisted surgery comprising:
a robotic guide assembly that includes a cutting guide;
a three-dimensional position tracking system for tracking a position of a first bone of a joint, a second bone of the joint, and the robotic guide assembly; and
a controller operatively in communication with the robotic guide assembly and the three-dimensional position tracking system, wherein the controller is configured to position the cutting guide adjacent one of the first and second bones of the joint, and then reposition the cutting guide adjacent the other one of the first and second bones of the joint, based on the tracked position of the first and/or second bones of the joint and the robotic guide assembly.

2. The robotic cutting guide system of claim 1, wherein the controller is further configured to adjust the position of the cutting guide in six degrees of freedom based on the tracked position of the first and/or second bones of the joint and the robotic guide assembly.

3. The robotic cutting guide system of claim 1, wherein the robotic guide assembly further includes a fixation bracket comprising:
a primary bracket; and
a connection bracket pivotably attached to the primary bracket.

4. The robotic cutting guide system of claim 3, wherein the connection bracket further includes a secondary pivot having an axis of rotation transverse to an axis of rotation of the primary bracket relative to the connection bracket.

5. The robotic cutting guide system of claim 1, wherein the robotic guide assembly further includes:
a positioning mechanism having a first arm with a first axis of rotation and a second arm having a second axis of rotation; and
a fixation bracket extending from the first arm, and
wherein the cutting guide extends from the second arm.

6. The robotic cutting guide system of claim 5, wherein the robotic guide assembly further includes a fixation bracket comprising:
a primary bracket; and
a connection bracket pivotable relative to the primary bracket about a primary axis of rotation, wherein the connection bracket includes pivot having a secondary axis of rotation transverse to the primary axis of rotation, and
wherein the primary axis of rotation and the secondary axis of rotation are both transverse to the first axis of rotation of the first arm and the second axis of rotation of the second arm of the positioning mechanism.

7. The robotic cutting guide system of claim 1, further comprising a joint distraction system for measuring distraction forces between first and second bones of a joint or determining predicted forces and/or predicted gaps between the first and second bones of the joint.

8. The robotic cutting guide system of claim 7, wherein the controller is configured to measure distraction forces between first and second bones of a joint or determine predicted forces and/or predicted gaps between the first and second bones of the joint based on a relative position between the first and second bones of the joint via the joint distraction system.

9. The robotic cutting guide system of claim 7, wherein the controller is configured to determine an optimal position for the cutting guide based on measured distraction forces between first and second bones of a joint or determined predicted forces and/or predicted gaps between the first and second bones of the joint.

10. The robotic cutting guide system of claim 7, wherein the controller is configured to update the position of the cutting guide adjacent the other one of the first and second bones of the joint, based on measured distraction forces between first and second bones of a joint or determined predicted forces and/or predicted gaps between the first and second bones of the joint.

11. The robotic cutting guide system of claim 1, wherein the controller is configured to determine an amount of deviation of the first and second bones from a datum reference and output a warning when the amount of deviation exceeds a predetermined criteria.

12. The robotic cutting guide system of claim 1, further comprising a pointer, and wherein the controller is configured to display on a display a surgical workflow when the pointer is positioned adjacent a predetermined device.

13. The robotic cutting guide system of claim 12, wherein the controller is configured to change a surgical workflow step when the pointer is positioned adjacent a predetermined device.

14. A method of resecting bones of a joint for an arthroplasty comprising:
using the robotic cutting guide system of claim 1:
attaching the robotic guide assembly having the cutting guide to a first bone of a joint;
tracking a position of the first bone of the joint, a second bone of the joint, and the robotic guide assembly;
positioning the cutting guide adjacent the first bone based on the tracked position of the first bone of the joint and the robotic guide assembly for resecting the first bone; and
repositioning the cutting guide adjacent the second bone based on the tracked position of the first and second bones of the joint and the robotic guide assembly for resecting the second bone.

15. The method of claim 14, wherein the repositioning of the cutting guide adjacent the second bone is completed while the robotic guide assembly is attached to the first bone.

16. The method of claim 14, further comprising adjusting the position of the cutting guide relative to one of the first and second bones of the joint up to six degrees of freedom.

17. The method of claim 14, further comprising, using a joint distraction system, measuring distraction forces between first and second bones of the joint or determining predicted forces and/or predicted gaps between the first and second bones of the joint based on a relative position between the first and second bones of the joint.

18. The method of claim 14, further comprising, using a joint distraction system, measuring distraction forces between first and second bones of the joint or determining predicted forces and/or predicted gaps between the first and second bones of the joint based on a relative position between the first and second bones of the joint after an implant has been implanted to one of the first and second bones of the joint.

19. The method of claim 14, further comprising, using a joint distraction system, measuring distraction forces between first and second bones of the joint or determining predicted forces and/or predicted gaps between the first and second bones of the joint based on a relative position between the first and second bones of the joint after resecting bone on only one of the first and second bones of the joint.

20. The method of claim 14, further comprising changing a step within a surgical workflow being performed by the robotic cutting guide system by moving a pointer tracked by the three-dimensional position tracking system to a predetermined position adjacent a predetermined device.
